(19) United States
(12) Patent Application Publication
(10) Pub. No.: US 2003/0212438 A1
(43) Pub. Date: Nov. 13, 2003

(54) Customization of medical device

(76) Inventors: Richard C. Nova, Kirkland, WA (US); Stephen L. Firman, Redmond, WA (US)

Correspondence Address: SHUMAKER & SIEFFERT, P. A., 8425 SEASONS PARKWAY, SUITE 105, ST. PAUL, MN, 55125, US

(21) Appl. No.: 10/141,683

(22) Filed: May 7, 2002

(51) Int. Cl. A61N 1/18

(52) U.S. Cl. 607/30

(57) ABSTRACT

A medical device user interface is customized for a particular type of user late in the production process or after delivery by using customization software loaded from an external source, such as a PC, a PDA or a network, or from a memory cartridge or replaceable component, e.g., a battery or a set of electrodes.

### TECHNICAL FIELD

The invention relates generally to medical devices and, more specifically, to medical devices used by diverse groups of users.

### BACKGROUND

Many medical devices are potentially used by medical personnel in different areas of the world. To maximize utility, user interfaces for such medical devices should be manageable by the intended user. For example, a user interface should present information in a language understood by medical personnel in the country in which the medical device is likely to be used. Some user interfaces are generic and are capable of comprehension by diverse users. Generic user interfaces, however, are limited in the amount and level of detail of information that can be conveyed.

Other medical devices have user interfaces that are customized for a specific group of users, e.g., users who speak a particular language. Some medical devices incorporate many elements that may need to be customized, including, for example, menus, user prompts, labels, and documentation. Each element that is customized may add to the time and cost of various aspects of product development, such as design engineering, manufacturing engineering, test engineering, and quality engineering. Furthermore, manufacturing costs may be increased as a result of a need to produce multiple versions of a product line in small quantities rather than larger quantities of a single version.

Customizing medical devices for diverse groups of users can lead to other disadvantages. Upgrading such devices to add features or replace obsolete components may be difficult due to the complexity and costs of implementation. Further, cost- and schedule-driven development compromises may result in confusing user interfaces or user interfaces that offer inadequate functionality.

### SUMMARY

In general, the invention facilitates customization of a medical device, such as an automated external defibrillator (AED), by incorporating customization features at late manufacturing stages or after manufacture. For example, a generic medical device can be customized for local language support via software downloaded from a personal computer, memory cartridge, docking station or network such as the Internet. Localization software can also be packaged with consumable items. The consumable items may be disposable or single-use items, such as batteries, electrodes, gloves or face shields. In this case, the consumable item may bear the same language as the language provided by the localization software upon installation in the medical device. In this manner, a generic medical device can be programmed just before use, instead of at the factory.

The invention may offer several advantages. Some implementations, for example, may allow multiple product lines to be produced as a single line of generic medical devices that are subsequently customized for use in a particular language. Such medical devices can be mass-produced in large quantities at a lower per-unit cost compared to medical devices that are customized earlier in the manufacturing process.

In one embodiment, the invention is directed to a method in which customization software is received in a medical device. A user interface is generated as a function of the customization software. The customization software may be received from a personal computer, personal digital assistant, docking station or a network such as the Internet. The customization software may also be received from a module inserted into the medical device, such as a memory cartridge or a disposable module, e.g., a battery or set of electrodes.

Other implementations include medical device systems that carry out these methods, as well as processor-readable media containing instructions that cause a processor to perform these methods. For example, in one embodiment, a medical device system includes a customization module containing customization software and a user interface module to generate a user interface in response to the customization software. The medical device system may also include a memory cartridge or disposable item containing the customization module. Alternatively, the customization module may be incorporated into the medical device system itself and loaded or enabled with customization software from an external source, such as a personal computer, personal digital assistant, docking station or network.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating a medical device system configured according to an embodiment of the invention.

FIG. 2 is a block diagram illustrating a medical device system customized via a computing device.

FIG. 3 is a block diagram illustrating a medical device system customized via a docking station.

FIG. 4 is a block diagram illustrating a medical device system customized via a memory cartridge.

FIG. 5 is a block diagram illustrating a medical device system customized via a disposable module.

FIG. 6 is a flow diagram illustrating customization of a medical device system.

### DETAILED DESCRIPTION

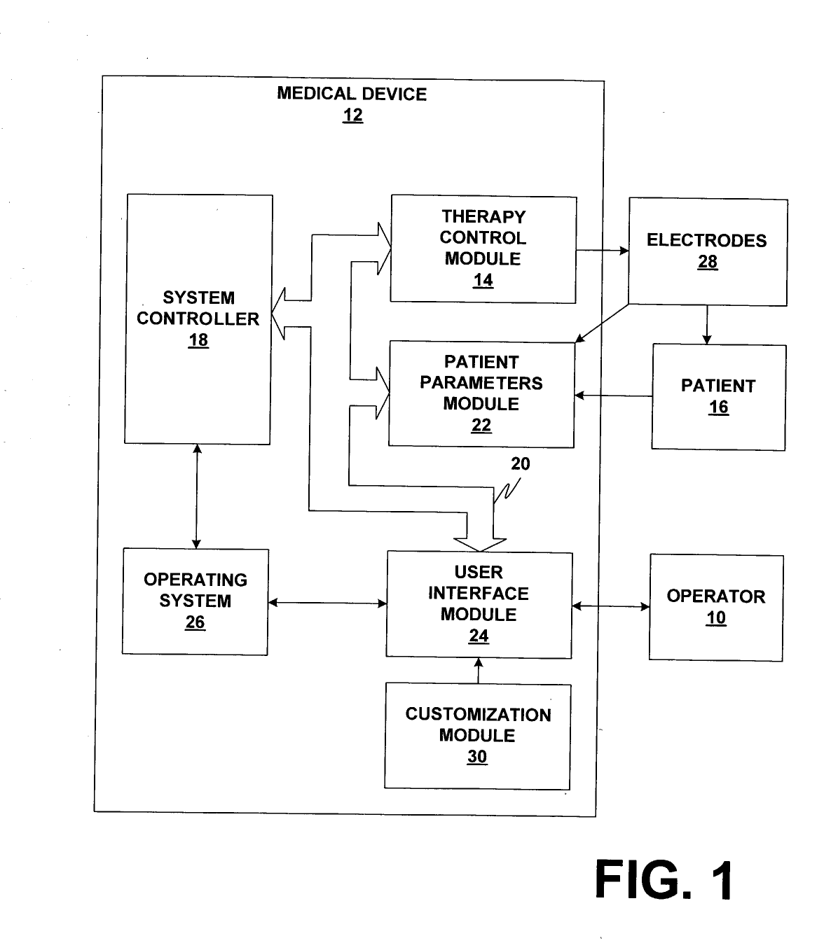

FIG. 1 is a block diagram illustrating a medical device system in which the invention may be practiced. When activated by an operator 10, a medical device 12 may administer a diagnostic and/or therapy regimen to a patient 16. Medical device 12 may be implemented, for example, as an automated external defibrillator (AED) that applies life-saving defibrillation shocks to patient 16. Medical device 12 is not limited to AED's, however, but includes medical devices that may deliver other forms of therapy, such as drug therapy, and/or medical devices that may perform diagnostic or monitoring functions, such as electrocardiogram (ECG) monitoring.

Operation of medical device 12 is controlled by a system controller 18 that is connected to a system bus 20. System controller 18 may be implemented as a microprocessor that communicates control and data signals with other components of medical device 12 via system bus 20. These components may include functional modules, such as therapy control module 14 or other diagnostic or therapy modules, a patient parameters module 22, and a user interface module 24.

In a medical device that delivers therapy, therapy control module 14 causes the therapy to be delivered to patient 16. For example, if medical device 12 is an AED, therapy control module 14 causes defibrillator electrodes 28 to deliver defibrillation shocks to patient 16 in response to control signals received from system controller 18 via system bus 20. Therapy control module 14 may include, for example, charging circuitry, a battery, and a discharge circuit. Any or all of these components can be controlled by system controller 18.

Patient parameters module 22 collects information from patient 16, including, for example, vital signs, non-invasive blood pressure (NIBP) measurements, and SpO₂ information. Other information relating to patient 16 may be collected by patient parameters module 22, including, but not limited to, ECG measurements, electroencephalogram measurements, invasive blood pressure measurements, temperature measurements, and ETCO₂ information. Information such as ECG measurements may be collected via electrodes 28. Other information may be collected via one or more sensors (not shown in FIG. 1).

User interface module 24 receives input from operator 10 and outputs information to operator 10 using any of a variety of input and output devices. For example, operator 10 may use a keyboard or touchpad to input commands to medical device 12 and receive prompts or other information via a display screen or LED indicators. A display device such as a display screen may present a user interface to operator 10. The display screen may present information in any of several forms such as prompts, menus, dialog boxes or graphical elements such as icons. The display screen may be implemented as a touch-screen display for both input and output. Medical device 12 may also communicate with operator 10 via an audio output device, e.g., a speaker that guides or prompts a user with a speech output in a particular language. In addition, user interface module 24 may print text reports or waveforms using a strip chart recorder or similar device. User interface module 24 may also interface with a rotary encoder device. The invention may be applied to all forms of input and output devices

User interface module 24 supplies input received from operator 10 or from patient 16 to an operating system 26 that controls operation of medical device 12 via system controller 18. Operating system 26 may be implemented as a set of processor-readable instructions that are executed by system controller 18. When medical device 12 is activated, operating system 26 may cause therapy control module 14 to deliver therapy or perform diagnostic functions. An AED, for example, may monitor ECG signals to determine whether patient 16 is exhibiting a shockable cardiac rhythm. When the patient 16 is exhibiting such a rhythm, the AED may administer therapeutic shocks to patient 16 via defibrillator electrodes 28.

An input or output device used in connection with user interface module 24 may present a user interface, such as a menu or dialog box, to operator 10. According to an embodiment of the invention, the user interface may be customized to the preferences of an anticipated user via a customization module 30. For example, customization module 30 may include localization software that presents a user interface in a language understood by a majority of people who live in the country or region in which medical device 12 is located. In addition, customization module 30 may present a user interface with particular metaphors, symbols or other graphical or textual objects that are understood within a particular country or region. Customization module 30 may also be configured to present a user interface to convey particular information that better conforms to regulatory or procedural norms in the country or region.

Customization module 30 may be programmed via any of a number of techniques. For example, customization module 30 may download localization software from a computing device such as a mainframe computer, personal digital assistant (PDA), personal computer (PC) or a network such as an intranet or the Internet. Downloading may take place at a late stage of manufacturing prior to delivery, or may take place after delivery. A technician or user may, for example, download localization software into customization module 30 after delivery, thereby customizing medical device 12 to a particular organization or user, or a local or regional distribution center. In another exemplary programming technique, customization module 30 may be programmed via a memory module that contains localization or other customization software.

Operating system 26 and user interfaces presented by input and output devices may be implemented as a set of processor-executable instructions stored in some form of processor readable media. Processor readable media can be any available media that can be accessed by medical device 12. By way of example, and not limitation, computer readable media may comprise computer storage media and communication media. Computer storage media includes volatile, and nonvolatile, removable and nonremovable media implemented in any method or technology for storage of information, such as computer readable instructions, data structures, program modules, or other data. Computer storage media includes, but is not limited to, random access memory (RAM), read only memory (ROM), EEPROM, flash memory or other memory technology, CD-ROM, digital versatile discs (DVD) or other optical storage, magnetic cassettes, magnetic tape, magnetic disk storage or other magnetic storage devices, or any other medium that can be used to store the desired information and that can be accessed by medical device 12. Communication media typically includes networks such as an intranet and the Internet, processor readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media, such as a wired network or other direct-wired connection, and wireless media, such as acoustic, RF, infrared, and other wireless media. Combinations of any of the above processor storage media and communication media are also included within the scope of processor-readable media.

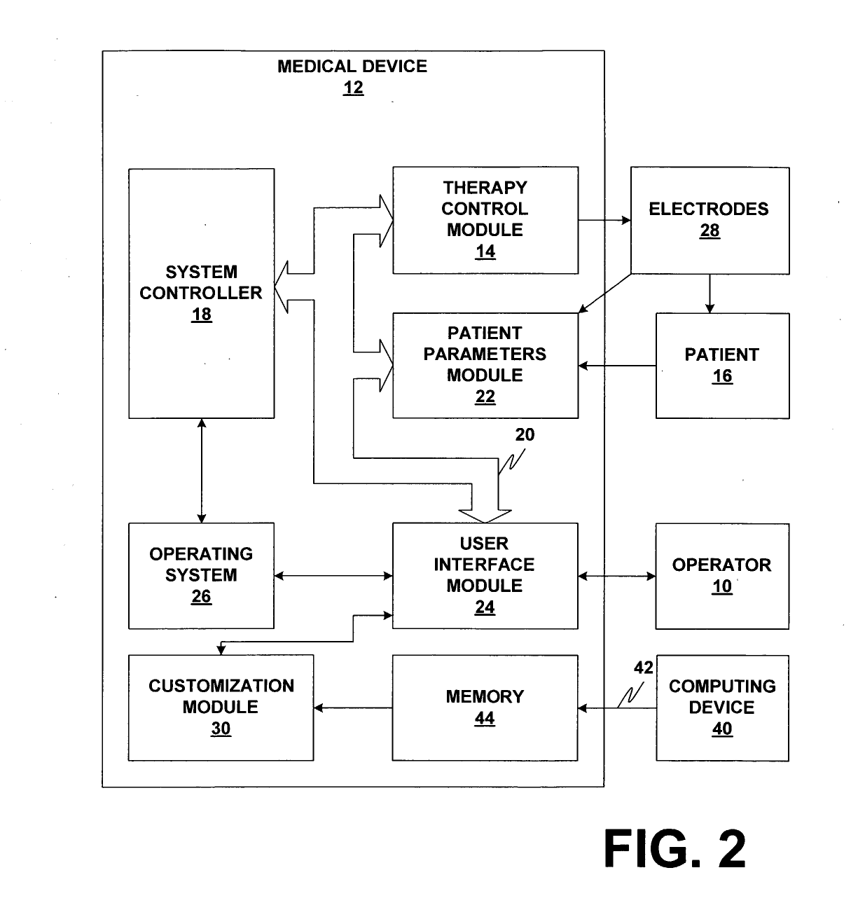

FIG. 2 is a block diagram illustrating a medical device system customized via a computing device 40. Medical device 12 communicates with a computing device 40 via a communication link 42. Communication link 42 may be a wired link, such as a USB cable, or a wireless link, such as an infrared or RF communication link. Although computing device 40 and communication link 42 are shown in FIG. 2 as external to medical device 12, the invention also encompasses embodiments in which the computing device and communication link are internal to medical device 12.

Computing device 40 may be any form of computing device, such as a PDA, a PC, a mainframe computer or a computer network. Computing device 40 may receive customization software from any computer readable medium such as the computer storage media and communication media described above. Customization software may include, for example, language localization software. Using communication link 42, medical device 12 may download the customization software from computing device 40. Medical device 12 may then store the customization software in a memory 44.

As noted above, user interface module 24 may interact with operator 10 through any of several input and output devices, such as a keyboard, display screen, a printing device or audio output device. User interface module 24 controls the presentation of the user interface. To present a customized user interface, user interface module 24 retrieves the customization software from memory 44 and renders the user interface according to the customization software. For example, the customization software may contain text blocks that are substituted for placeholders in a user interface template stored in user interface module 24. Combining the template with the text blocks allows user interface module 24 to generate a user interface that is customized for the needs of a particular type of user, such as an operator who understands a specific language or an operator with a certain level of technical proficiency.

In some implementations, the customization software may include multiple groups of text blocks for customizing the user interface to multiple types of users. For example, the customization software may contain text blocks for language localization of medical device 12 to several languages. As another example, the customization software may allow operator 16 to select a level of detail of prompts, e.g., terse or verbose. In this manner, the operation of medical device 12 can be facilitated for a variety of potential users.

The customization software may also customize medical device 12 by enabling and/or disabling pre-programmed functions. In some implementations, for example, medical device 12 may be pre-programmed with multiple groups of text blocks in multiple languages. The customization software, instead of supplying the text blocks in one or more languages, may enable user interface module 24 with one language and disable other languages. In another illustrative implementation, the customization software may, for example, enable an enhanced interface for users having a high level of technical proficiency, and disable a more simplified interface.

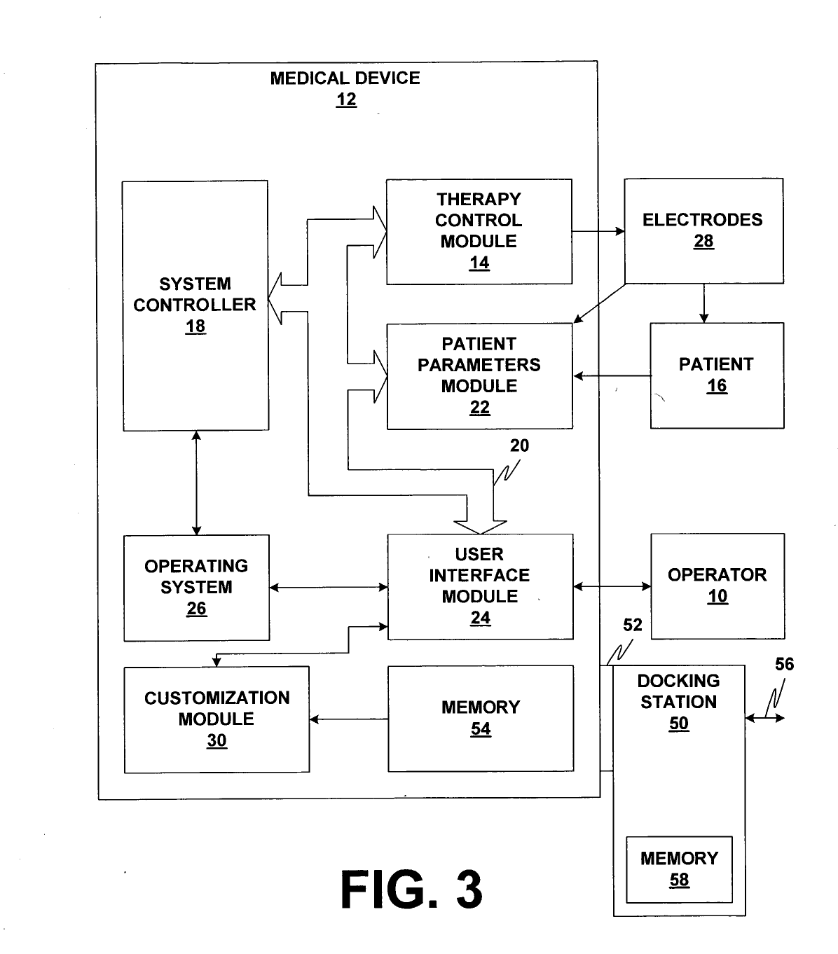

FIG. 3 is a block diagram illustrating a medical device system customized via a docking station 50. A docking station port 52 integrated in medical device 12 communicates with docking station 50. Docking station 50 may receive language localization software or other customization software from the Internet, a computing device or any other external source via a communication link 56. Communication link 56 may be a wired link, such as a USB cable, or a wireless link, such as an infrared or RF communication link. Alternatively, docking station 50 may have customization software pre-loaded into a memory 58. Medical device 12 subsequently receives the customization software from docking station 50 through docking station port 52 and stores the customization software in a memory 54.

In some embodiments, docking station 50 may be used to customize several generic medical devices 12. Memory 58 may store language localization software for multiple languages. A technician or other personnel may select, for each generic medical device 12, which language localization software to load from memory 58 and program into medical device 12. If docking station 50 instead receives language localization software via communication link 56, the person programming medical device 12 can select which language localization software to download via communication link 56 and load into memory 54 of medical device 12.

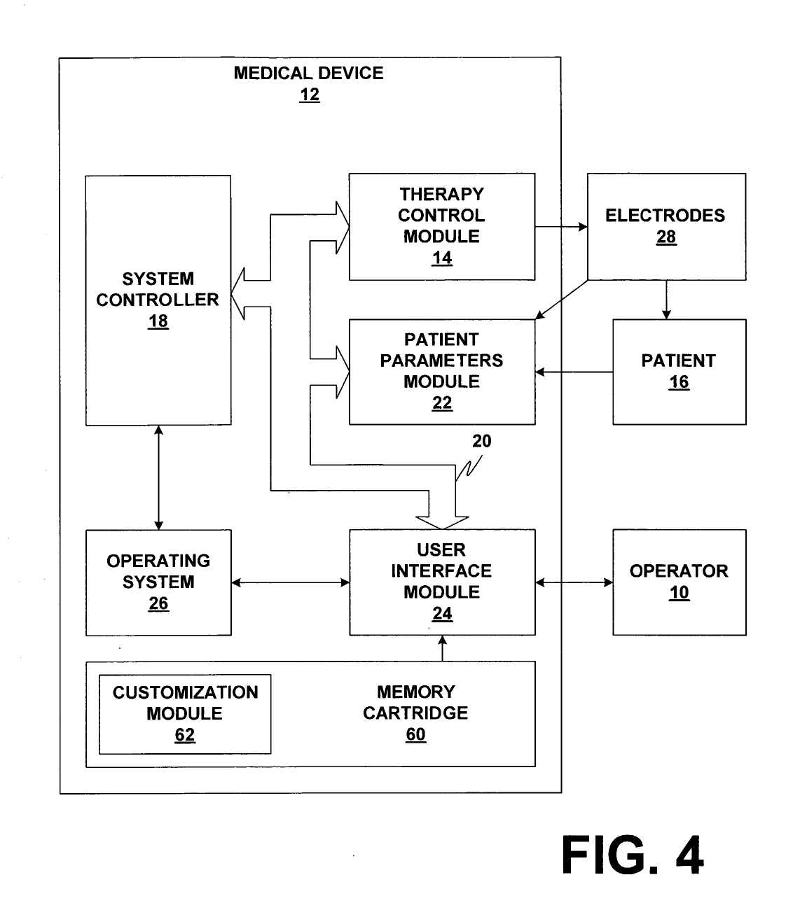

FIG. 4 is a block diagram illustrating a medical device system customized via a memory cartridge 60 inserted into medical device 12. Memory cartridge 60 contains a customization module 62 that includes customization software, such as language localization software. Memory cartridge 60 may also contain memory that may store text blocks and other data that is accessed in connection with the customization software.

When memory cartridge 60 is inserted into medical device 12, user interface module 24 reads the customization software from memory cartridge 60 and causes an input or output device to render a user interface as specified by the customization software. Implementing customization module 62 on a removable memory cartridge may reduce costs of manufacture by facilitating production of larger quantities of generic medical devices 12, which are subsequently customized by insertion of appropriate memory cartridges 60.

In addition to providing language localization capabilities, memory cartridge 60 can also implement other forms of customization. In one embodiment of the invention, optional accessories may be distributed with memory cartridges 60 that contain customization modules 62 for customizing the user interface for use with the accessories. Optional accessories may include a specialized input/output device such as a sensor or transducer. An optional pulse oximetry transducer, for example, when coupled to an AED, may enable the AED to perform pulse oximetry and to display data obtained via pulse oximetry. The pulse oximetry transducer may be bundled with customization software that supports an interface between the AED and the transducer, and/or the transducer may enable pre-programmed oximetry functions in the AED. Another form of optional accessory may be a training module that, when coupled to medical device 12, converts medical device 12 into training equipment.

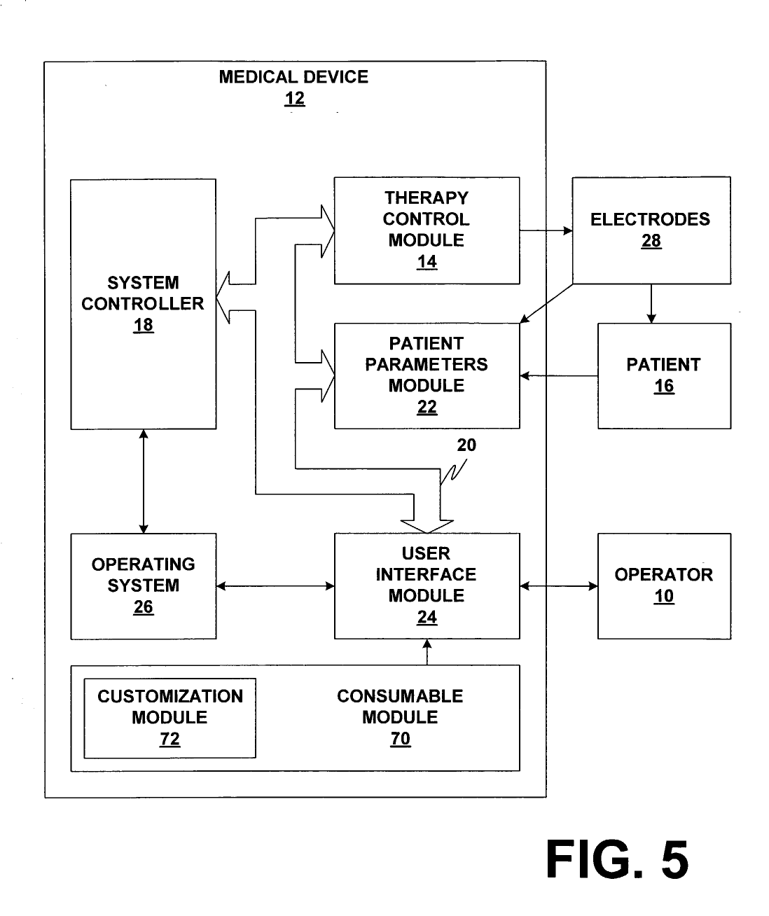

FIG. 5 is a block diagram illustrating a medical device system customized via a consumable module 70, such as a battery or a set of electrodes. Consumable module 70 may be disposable, as in the case of a battery, or may be reusable. Consumable module 70 contains a customization module 72 that includes customization software, such as language localization software. Consumable module 70 may also contain memory that may store text blocks and other data that is accessed in connection with the customization software. Alternatively, this data may be stored in a memory (not shown) integrated with medical device 12.

When consumable module 70 is inserted into medical device 12, user interface module 24 reads the customization software from consumable module 70 and causes an input or output device to render a user interface as specified by the customization software. Implementing customization module 72 on a removable component, such as a battery or a set of electrodes, may reduce costs of manufacture by facilitating production of larger quantities of generic medical devices 12, which are subsequently customized by insertion of appropriate consumable modules 70.

In addition to providing language localization capabilities, consumable module 70 can also implement other forms of customization. For example, some consumable modules 70, such as optional accessories that provide enhanced functionality, may contain customization modules 72 for customizing the user interface for use with the consumable modules 70. As described above, an input/output device such as a sensor or transducer may provide enhanced functionality and may customize medical device 12 and user interface module 24 accordingly. In addition, inclusion of customization module 72 in single-use or periodically replaceable consumable module 70 enables customization module 72 to include software upgrades, such as software enhancements or patches.

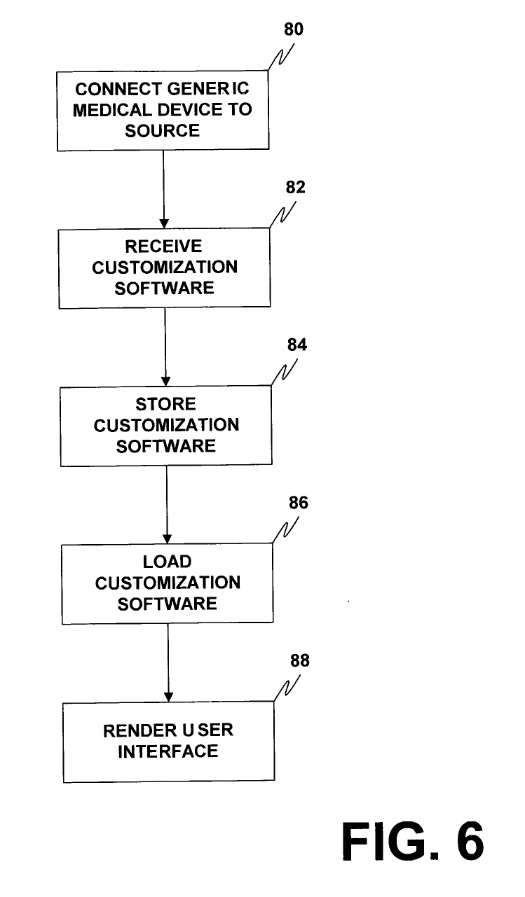

FIG. 6 is a flow diagram illustrating customization of a medical device system. At some point in the manufacturing process or after delivery, a generic medical device 12 is connected to a source of customization software (80). This source may be a computing device or other external source connected by a communication link, such as computing device 40 of FIG. 2 or docking station 50 of FIG. 3. Alternatively, the source may be inserted into medical device 12, as shown in FIGS. 4 and 5. Medical device 12 then receives the customization software from the source (82) and stores the customization software in a memory (84). As a result, medical device 12 is language-localized or otherwise customized. Subsequently, during normal use, medical device 12 loads the customization software (86) and renders a user interface according to the customization software (88). By way of example, the customization software may be combined with a generic medical device user interface template to generate a user interface in a particular language. The customization software can also generate the user interface without using a template.

Customizing the user interface presented by medical device 12 late in the manufacturing process or after delivery may decrease production costs by enabling production of large quantities of a single product line, i.e., generic medical devices, rather than smaller quantities of multiple product lines. As a result, design and manufacturing costs may be reduced. Costs associated with testing and quality control may also be reduced. Separating the language localization or customization process from other phases in the production process also facilitates product upgrades by lowering implementation costs. In addition, time-to-market delays can be reduced. A product may be quickly brought to market in one locality, for example, and may later be customized for other localities.

Various embodiments of the invention have been described. Modifications can be made without departing from the spirit and scope of the invention. For example, as described above, the invention is not limited to language localization, but can also be used for customizing other aspects of a medical device user interface. The invention may be used in AEDs as well as other types of defibrillators. The invention is not limited to defibrillators, however, but may also be used in other types of medical devices, including, but not limited to, defibrillator/pacemakers and therapy devices for other medical conditions, such as stroke and respiratory conditions. The invention may be applied with medical devices that provide therapy, medical devices that provide diagnosis, medical devices that provide monitoring, and medical devices that provide any combination thereof. These and other embodiments are within the scope of the following claims.

1. A method comprising:
receiving customization software in a medical device from a device external to the medical device; and
generating a user interface as a function of the customization software.

2. The method of claim 1, further comprising receiving the customization software from a computing device.

3. The method of claim 2, wherein the computing device comprises at least one of a mainframe computer, a personal computer, a personal digital assistant and a network.

4. The method of claim 1, further comprising receiving the customization software using a docking station.

5. The method of claim 1, further comprising receiving the customization software from a removable memory cartridge.

6. The method of claim 1, further comprising receiving the customization software from a consumable module.

7. The method of claim 6, wherein the consumable module comprises at least one of a battery and a set of electrodes.

8. The method of claim 1, wherein the customization software comprises language localization software.

9. The method of claim 1, wherein the customization software is received after manufacture of the medical device.

10. The method of claim 1, wherein the user interface comprises at least one of a text element, a graphical element, and a speech output.

11. A medical device system comprising:
a customization module containing customization software received from a device external to the medical device system; and
a medical device comprising a user interface module to generate a user interface for the medical device in response to the customization software.

12. The medical device system of claim 11, wherein the customization module receives the customization software from a computing device.

13. The medical device system of claim 12, wherein the computing device comprises at least one of a mainframe computer, a personal computer, a personal digital assistant and a network.

14. The medical device system of claim 11, wherein the customization software is received using a docking station.

15. The medical device system of claim 11, further comprising a memory cartridge containing the customization software.

16. The medical device system of claim 11, further comprising a consumable module containing the customization software.

17. The medical device system of claim 16, wherein the consumable module comprises at least one of a battery and a set of electrodes.

18. The medical device system of claim 11, wherein the customization software comprises language localization software.

19. The medical device system of claim 11, wherein the customization software is received after manufacture of the medical device system.

20. The medical device system of claim 11, wherein the user interface comprises at least one of a text element, a graphical element, and a speech output.

21. A processor-readable medium containing processor executable instructions for:
receiving customization software in a medical device from a device external to the medical device; and
generating a user interface in the medical device as a function of the customization software.

22. The processor-readable medium of claim 21, wherein the customization software is received from a computing device.

23. The processor-readable medium of claim 22, wherein the computing device comprises at least one of a mainframe computer, a personal computer, a personal digital assistant and a network.

24. The processor-readable medium of claim 21, wherein the customization software is received using a docking station.

25. The processor-readable medium of claim 21, wherein the customization software is received from a removable memory cartridge.

26. The processor-readable medium of claim 21, wherein the customization software is received from a disposable module.

27. The processor-readable medium of claim 26, wherein the disposable module comprises at least one of a battery and a set of electrodes.

28. The processor-readable medium of claim 21, wherein the customization software comprises language localization software.

29. The processor-readable medium of claim 21, wherein the customization software is received after manufacture of the medical device.

30. The processor-readable medium of claim 21, wherein the user interface comprises at least one of a text element, a graphical element, and a speech output.
